(19) United States
(12) Patent Application Publication
(10) Pub. No.: US 2021/0238583 A1
(43) Pub. Date: Aug. 5, 2021

(54) NUCLEIC ACID ISOLATION AND RELATED METHODS

(71) Applicant: Cepheid, Sunnyvale, CA (US)

(72) Inventors: Alex I. KUTYAVIN, Sunnyvale, CA (US); Oliver Z. NANASSY, Sunnyvale, CA (US); Dmitri SERGUEEV, Sunnyvale, CA (US); Alexander A. GALL, Sunnyvale, CA (US)

(73) Assignee: Cepheid, Sunnyvale, CA (US)

(21) Appl. No.: 17/269,198

(22) PCT Filed: Aug. 16, 2019
(86) PCT No.: PCT/US2019/046912

§ 371 Date: Feb. 17, 2021

Related U.S. Application Data
(60) Provisional application No. 62/765,149, filed on Aug. 17, 2018.

(51) Int. Cl. C12N 15/10; C12Q 1/6806

(52) U.S. Cl. CPC C12N 15/1006; C12Q 1/6806

(57) ABSTRACT

Solid supports modified with pectins derivatives are provided. The solid supports are useful in nucleic acid isolation, separation, and detection methods.

### CROSS-REFERENCE(S) TO RELATED APPLICATION(S)

This application claims the benefit of U.S. Provisional Application No. 62/765,149, filed Aug. 17, 2018, which is incorporated herein by reference in its entirety.

### FIELD OF THE INVENTION

The present invention relates to solid supports comprising modified pectins and methods of their use.

### BACKGROUND

Molecular diagnostic assays that utilize amplification and/or detection of nucleic acids by various automated analytical techniques, such as polymerase chain reaction (PCR), provide rapid and accurate results in less time compared to traditional diagnostic methods and can be easily automated. However, in order to perform molecular diagnostic analysis of biological samples, nucleic acids have to be isolated from the biological materials to remove components that can affect the accuracy of the assay, e.g., by inhibiting the polymerase activity. Even though a variety of methods for nucleic acid extraction exists, currently available methods generally involve lengthy steps and are not easily amenable to automation. Thus, preparation of nucleic acid samples prior to amplification and detection of specific targets is the most challenging step of molecular diagnostics.

Simple and rapid methods of nucleic acid isolation that do not require extensive sample processing and that can be adapted to clinical laboratory automation are needed for producing quality nucleic acids free of inhibitors of amplification. There is a need for reagents that can facilitate isolation of nucleic acids from nucleic acid-containing biological samples in a manner compatible with fast, automated nucleic acid detection methods. The present invention fulfills this need and provides further related advantages.

### SUMMARY

In one aspect, provided herein a solid support comprising a plurality of modified pectin molecules covalently bound to the solid support. In some embodiments, the modified pectin comprises a plurality of amino groups. In some embodiments, the modified pectin is an amidated pectin. In some embodiments, the amidated pectin comprises one or more units represented by Formula:

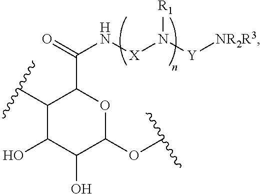

an isomer, a salt, or a tautomer thereof,

wherein

n is 0, 1, 2, or 3;

R¹ is H or C₁-C₃ alkyl;

X, at each occurrence, is independently C₂-C₄ alkylene or C₄-C₆ heteroalkylene;

Y is a C₂-C₃ alkylene or C₄-C₆ heteroalkylene; and

R² and R³ are independently H or C₁-C₃ alkyl.

In some embodiments, the amidated pectin is a pectin amidated with a C₄-C₂₀ polyamine. In some embodiments, the polyamine is ethylenediamine, putrescine, cadaverine, spermine, or spermidine.

In some embodiments, the amidated pectin comprises one or more units having the structure:

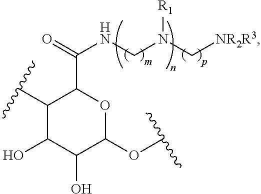

an isomer, a salt, or a tautomer thereof,

wherein

n is 0, 1, 2, or 3;

m is 2, 3, or 4;

p is 2, 3, or 4; and

R¹, R², and R³ are independently H or C₁-C₃ alkyl.

In some embodiments, the amidated pectin comprises one or more units having the structure:

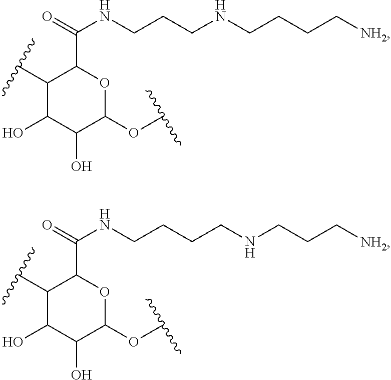 [SMILES: *OC1OC(C(=O)NCCCNCCCCN)C(*)C(O)C1O ; *OC1OC(C(=O)NCCCCNCCCN)C(*)C(O)C1O]

an isomer, a salt, or a tautomer thereof.

In some embodiments, the amidated pectin is amidated citrus pectin or amidated apple pectin. In some embodiments, the amidated pectin has a molecular weight between about 4,000 Da and about 500,000 Da, between about 5,000 Da and about 300,000 Da, between about 100,000 Da and about 300,000 Da, or between about 50,000 Da and about 200,000 Da.

In some embodiments, the solid support comprises a material selected from polystyrene, glass, ceramic, polypropylene, polyethylene, silica, zirconia, titania, alumina, polycarbonate, latex, polyethersulfone, PMMA, carboxymethylcellulose, zeolite, and cellulose.

In some embodiments, the solid support is a magnetic bead, a glass bead, polystyrene bead, a polystyrene filter, a polycarbonate filter, a polyethersulfone, or a glass filter.

In another aspect, provided herein is a method for isolation of a nucleic acid from a nucleic-acid containing sample, comprising:

(a) contacting the sample with a solid support disclosed herein thereby binding the nucleic acid to the solid support;

(b) optionally washing the nucleic acid bound to the solid support; and

(c) eluting the nucleic acid from the solid support with an eluting reagent.

In some embodiments, the eluting agent comprises ammonia or an alkali metal hydroxide. In some embodiments, the eluting agent has a pH of above about 9, above about 10, or above about 11. In some embodiments, the eluting reagent has a pH of about 9 to about 12, about 9.5 to about 12, about 10 to about 12, or about 9 to about 11. In some embodiments, the eluting reagent comprises a polyanion. In some embodiments, the polyanion is carrageenan or a carrier nucleic acid. In some embodiments, the eluting agent comprises a polyanion and a base, e.g., an alkali hydroxide. In some embodiments, the eluting agent comprises i-carrageenan and KOH.

In some embodiments, the method comprises contacting the sample with a lysis solution prior to contacting the sample with the solid support, thereby releasing nucleic acids into solution. In some embodiments, the lysis solution comprises a chaotropic agent. In some embodiments, the chaotropic agent is selected from guanidinium thiocyanate, guanidinium hydrochloride, alkali perchlorate, alkali iodide, urea, formamide, or combinations thereof. In some embodiments, the chaotropic agent is guanidinium thiocyanate or guanidinium hydrochloride. In some embodiments, the lysis solution comprises a salt. In some embodiments, the salt is sodium chloride or calcium chloride. In some embodiments, the lysis solution does not contain a chaotropic agent. In some embodiments, the lysis solution comprises a buffering agent. In some embodiments, the buffering agent is Tris. In some embodiments, the lysis solution comprises a surfactant. In some embodiments, the lysis solution comprises a defoaming agent.

In some embodiments, contacting the sample with a solid support is done without the presence of a chaotropic reagent.

In some embodiments, the sample is selected from blood, plasma, serum, semen, tissue biopsy, urine, stool, saliva, smear preparation, bacterial culture, cell culture, viral culture, PCR reaction mixture, or in vitro nucleic acid modification reaction mixture. In some embodiments, the tissue biopsy is a paraffin-embedded tissue. In some embodiments, the nucleic acid comprises genomic DNA. In some embodiments, the nucleic acid comprises total RNA. In some embodiments, the nucleic acid comprises microbial nucleic acid or viral nucleic acid. In some embodiments, the viral nucleic acid is HBV DNA. In some embodiments, the nucleic acid is a circulating nucleic acid.

In some embodiments, the method is performed in an automated cartridge.

In another aspect, provided herein is a method for detecting a nucleic acid in a sample, comprising:

(a) contacting a nucleic acid-containing sample with a solid support disclosed herein thereby binding the nucleic acid to the solid support;

(b) optionally washing the nucleic acid bound to the solid support;

(c) eluting the nucleic acid; and

(d) detecting the nucleic acid.

In some embodiments, detecting the nucleic acid comprises amplification of the nucleic acid by polymerase chain reaction (PCR). In some embodiments, the polymerase chain reaction is a nested PCR, an isothermal PCR, or RT-PCR.

In another aspect, provided herein is a separating material for chromatography comprising a solid support comprising an amidated pectin covalently bonded thereto.

In some embodiments, the amidated pectin has one or more units represented by formula:

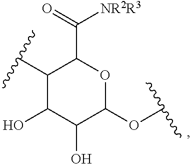

an isomer, a salt, or a tautomer thereof,

R² and R³ are independently selected from H, optionally substituted C₁-C₆ alkyl, optionally substituted C₃-C₆ cycloalkyl, and optionally substituted C₂-C₂₀ heteroalkyl.

In some embodiments, the solid support is silica, alumina, titania, zirconia, or a hybrid silica material.

### DETAILED DESCRIPTION OF THE INVENTION

In one aspect, provided herein are solid supports for purification of nucleic acids from nucleic-acid containing samples, comprising one or more molecules of modified pectins covalently bound to the surface. In some embodiments, the modified pectin comprises a plurality of amino groups. In some embodiments, the modified pectin is an amidated pectin. As used herein, the term “solid support” refers to any substrate including paramagnetic particles, gels, controlled pore glass, magnetic beads, microspheres, nanospheres, capillaries, filter membranes, columns, cloths, wipes, paper, flat supports, multi-well plates, porous membranes, porous monoliths, wafers, combs, or any combination thereof. Solid supports can comprise any suitable material, including but not limited to glass, silica, titanium oxide, iron oxide, ethylenic backbone polymers, polypropylene, polyethylene, polystyrene, ceramic, cellulose, nitrocellulose, and divinylbenzene. Preferably, solid support comprises a material selected from polystyrene, glass, ceramic, polypropylene, polyethylene, silica, polycarbonate, latex, PMMA, zeolite, polyethersulfone, carboxymethylcellulose, cellulose, and combinations thereof. In some embodiments, the solid support is not a not a pectin, e.g., an unmodified pectin or a modified pectin.

In some embodiments, the solid support is a magnetic bead, a glass bead, a polystyrene bead, a polystyrene filter, a polycarbonate filter, a polyethersulfone filter, or a glass filter. Preferably, the materials suitable for the preparation of the solid supports disclosed herein have low non-specific binding, e.g., in the absence of pectin modifications described herein, these materials do not bind nucleic acids, proteins, or other components of the sample from which isolation of nucleic acid is desired.

Modified Pectins

In some embodiments, the modified pectins are amidated pectins. Pectins are naturally occurring complex polysaccharides typically found in plant cell walls. Pectins comprise an alpha 1-4 linked polygalacturonic acid backbone intervened by rhamnose residues and modified with neutral sugar side chains and non-sugar components such as acetyl, methyl, and ferulic acid groups. The galacturonic acid residues in pectin are partly esterified and present as the methyl esters. The degree of esterification is defined as the percentage of carboxyl groups esterified. Pectins with a degree of esterification, e.g., above 50%, are classified as high methyl ester (“HM”) pectins or high ester pectins, and pectins with a degree of esterification lower than 50% are referred to as low methyl ester (“LM”) pectins or low ester pectins. Most pectin found in fruits and vegetables are HM pectins.

As used herein, “amidated pectin” refers to any naturally occurring pectin that has been structurally modified, e.g., by chemical, physical, or biological (including enzymatic) means, or by some combination thereof, wherein some of the ester or acid groups have been converted to amide groups. Amidated pectins can be prepared by contacting unmodified pectin with a solution of a suitable amine thereby converting the ester groups of the unmodified pectin to amides.

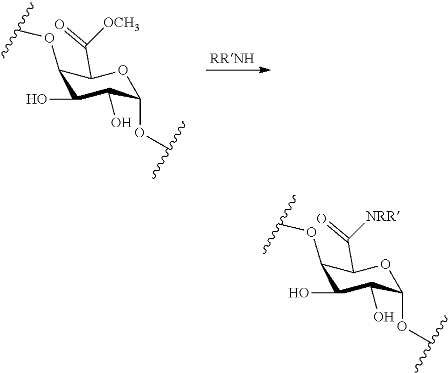

Alternatively, unmodified pectin or hydrolyzed pectin, including partially hydrolyzed pectin, can be reacted with an amine in the presence of a suitable coupling agent to form amidated pectin. Non-limiting examples of suitable coupling agents include carbodiimide coupling agents such as DCC and EDCI, and phosphonium and imonium type reagents such as BOP, PyBOP, PyBrOP, TBTU, HBTU, HATU, COMU, and TFFH.

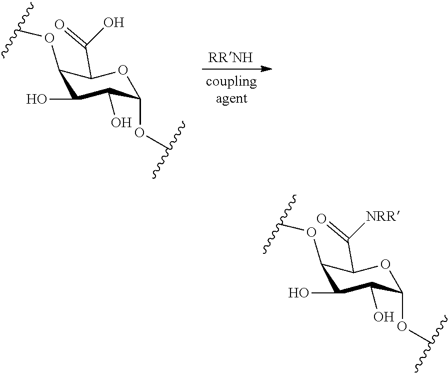

In some embodiments, the modified pectin is a modified pectin obtained by reductive amination of a periodate-oxidized pectin. Methods of reductive amination of carbohydrates, such as pectins, are known in the art.

A modified pectin can be obtained by any of the methods described herein from an unmodified pectin. Particularly useful starting materials for modified pectin synthesis are apple and citrus pectins. In some embodiments, the starting pectins have molecular weights from about 4,000 Da to about 500,000 Da, from about 5,000 Da to about 300,000 Da, from about 10,000 Da to about 150,000 Da, or from about 10,000 Da to about 100,000 Da.

In some embodiments, the amidated pectin comprises a plurality of uronic acid units and one or more additional monomeric units. Uronic acids include sugar acids comprising both carbonyl (e.g., aldehyde or keto group) and carboxylic acid (—COOH) functional groups. Typically, urionic acids are derived from sugars in which the terminal hydroxyl group has been oxidized to a carboxylic acid and are generally named according to their parent sugars, for example, a glucuronic acid is the uronic acid derived from glucose. Uronic acids derived from hexoses are known as hexuronic acids, and uronic acids derived from pentoses are known as penturonic acids.

In some embodiments, in addition to one or more uronic acid units, the amidated pectin further comprises one or more units selected from:

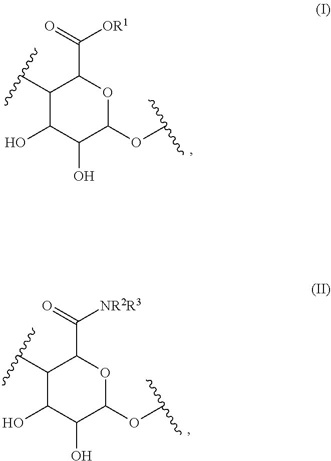

their isomers, salts, tautomers, and combinations thereof,

wherein R¹ is selected from optionally substituted C₁-C₈ alkyl, optionally substituted C₃-C₈ cycloalkyl, optionally substituted C₃-C₈ heterocycloalkyl, and optionally substituted C₂-C₂₀ heteroalkyl; and

R² and R³ are independently selected from H, optionally substituted C₁-C₆ alkyl, optionally substituted C₃-C₆ cycloalkyl, and optionally substituted C₂-C₂₀ heteroalkyl.

In some embodiments, R³ is an optionally substituted C₁-C₆ alkyl In some embodiments, R³ is an optionally substituted C₄-C₂₀ heteroalkyl, for example, an short PEG chain optionally substituted with one or more amino groups. In some embodiments,

In some embodiments, each of R¹, R², and R³ comprises no more than one amino group. In some embodiments, each of R¹, R², and R³ does not comprise an amino group. In some embodiments, each of R² and R³ comprise one or more amino groups. In some embodiments, R² is H and R³ is an optionally substituted C₄-C₂₀ heteroalkyl, for example, a polyamine or an oligomeric ethylene glycol comprising 2-6 ethylene glycol units, optionally substituted with one or more amino groups.

In some embodiments, R¹ is methyl, ethyl, or propyl. In some embodiments, R² and R³ are both H. In some embodiments, R² is H and R³ is an optionally substituted C₁-C₈ alkyl. In some embodiments, R² is H and R³ is H, CH₃ CH₂CH₂NH₂, CH₂CH₂N(CH₃)₂, CH₂CH₂OH, or CH₂CH₂NHCH₂CH₂NH₂. In some embodiments, R² and R³ are both CH₃.

In some embodiments, the amidated pectin further comprises one or more units of Formula (III):

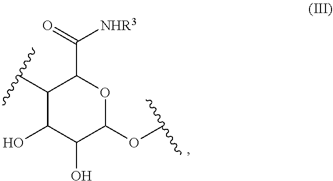

or

an isomer, a salt, a tautomer, or a combination thereof, wherein:

R³ is H, CH₃, CH₂CH₂NH₂, CH₂CH₂N(CH₃)₂, CH₂CH₂OH, (CH₂)₂O(CH₂)₂NH₂, or CH₂CH₂NHCH₂CH₂NH₂.

It is understood that if a polysaccharide comprises two or more units of Formula (II) or (III), their R³ can be the same or different within the polysaccharide.

In some embodiments, the amidated pectins disclosed herein comprise one or more monomeric units having at least one amino group. In some embodiments, the amidated pectins comprise one or more monomeric units having the structure of Formula VI:

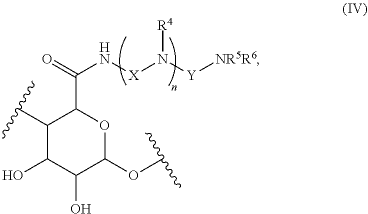

an isomer, a salt, a tautomer, or a combination thereof, wherein:

n is 0, 1, 2, or 3;

R⁴ is H or C₁-C₃ alkyl;

X, at each occurrence, is independently C₂-C₄ alkylene or C₄-C₆ heteroalkylene;

Y is a C₂-C₃ alkylene or C₄-C₆ heteroalkylene; and

R⁵ and R⁶ are independently H or C₁-C₃ alkyl.

In some embodiments, the amidated pectins disclosed herein comprise one or more monomeric units having the structure of Formula V:

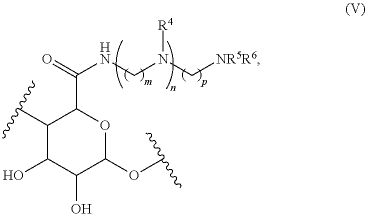

an isomer, a salt, a tautomer, or a combination thereof, wherein:

n is 0, 1, 2, or 3;

m, at each occurrence, is independently 2, 3, or 4;

p is 2, 3, or 4;

R⁴ is H or C₁-C₃ alkyl; and

R⁵ and R⁶ are independently H or C₁-C₃ alkyl.

In some embodiments, the amidated pectin comprises one or more monomeric units comprising a primary amino group. In some embodiments, the amidated pectin comprises one or more monomeric units comprising a quarternary ammonium group. In some embodiments, the amidated pectin is amidated with a polyamine. As used herein, a polyamine is a compound comprising two or more amino groups. Polyamines that can be used for modification of pectins of the solid supports disclosed herein include both synthetic polyamines and naturally occurring polyamines, e.g., spermidine, spermine, putrescine. In some embodiments, the polyamine is selected from the group consisting of spermine, spermidine, cadaverine, ethylenediamine, and putrescine. In some embodiments, the polyamine is spermine or spermidine.

In some embodiments, the amidated pectin comprises one or more units having the structure of Formula VI, Formula VII, or Formula VIII, including their isomers, salts, and tautomers:

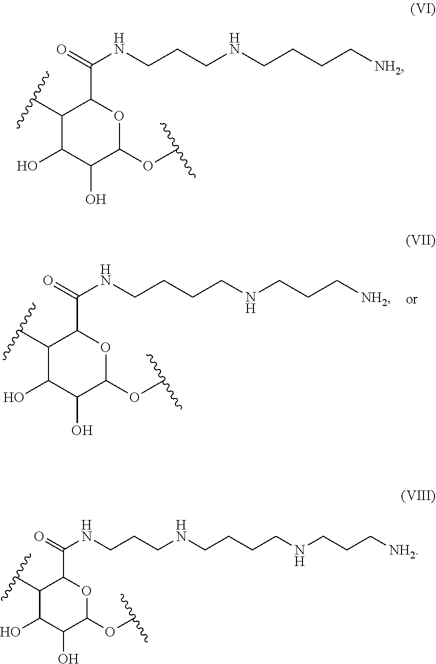 [SMILES: *OC1OC(C(=O)NCCCNCCCCN)C(*)C(O)C1O ; *OC1OC(C(=O)NCCCCNCCCN)C(*)C(O)C1O ; *OC1OC(C(=O)NCCCNCCCCNCCCN)C(*)C(O)C1O]

In some embodiments, the amidated pectins comprise a plurality of additional monomeric units represented by the structure of Formulae I-VIII. As used herein, the term “plurality” means more than one. For example, a plurality of monomeric units means at least two monomeric units, at least three monomeric units, or at least monomeric units, and the like. If an embodiment of the present invention comprises more than one monomeric units, they may also be referred to as a first monomeric unit, a second monomeric unit, a third monomeric unit, etc.

As used herein, the terms “alkyl,” “alkenyl,” and “alkynyl” include straight-chain, branched-chain, and cyclic monovalent hydrocarbyl radicals, and combinations thereof, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms, it can be represented as 1-10C, C1 -C10, C₁-C₁₀, C₁₋₁₀, or C1-10. The term “heteroalkyl,” “heteroalkenyl,” and “heteroalkynyl,” as used herein, mean the corresponding hydrocarbons wherein one or more chain carbon atoms have been replaced by a heteroatom. Exemplary heteroatoms include N, O, S, and P. When heteroatoms are allowed to replace carbon atoms, for example, in heteroalkyl groups, the numbers describing the group, though still written as e.g. C3-C10, represent the sum of the number of carbon atoms in the cycle or chain plus the number of such heteroatoms that are included as replacements for carbon atoms in the cycle or chain being described.

A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl, and alkynyl groups can be optionally substituted to the extent that such substitution makes sense chemically. Typical substituents include, but are not limited to, halogens (F, Cl, Br, I), ═O, ═NCN, ═NOR, ═NR, OR, NR₂, SR, SO₂R, SO₂NR₂, NRSO₂R, NRCONR₂, NRC(O)OR, NRC(O)R, CN, C(O)OR, C(O)NR₂, OC(O)R, C(O)R, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halogens (F, Cl, Br, I), ═O, ═NCN, ═NOR′, ═NR′, OR′, NR′₂, SR′, SO₂R′, SO₂NR′₂, NR′SO₂R′, NR′CONR′₂, NR′C(O)OR′, NR′C(O)R′, CN, C(O)OR′, C(O)NR′₂, OC(O)R′, C(O)R′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl, and alkynyl groups can also be substituted by C1-C8 acyl, C2-C8 heteroacyl, C6-C10 aryl or C5-C10 heteroaryl, each of which can be substituted by the substituents that are appropriate for the particular group.

While “alkyl” as used herein includes cycloalkyl and cycloalkylalkyl groups, the term “cycloalkyl” is used herein to describe a carbocyclic non-aromatic group that is connected via a ring carbon atom, and “cycloalkylalkyl” is used to describe a carbocyclic non-aromatic group that is connected to the molecule through an alkyl linker. Similarly, “heterocyclyl” is used to identify a non-aromatic cyclic group that contains at least one heteroatom as a ring member and that is connected to the molecule via a ring atom, which may be C or N; and “heterocyclylalkyl” can be used to describe such a group that is connected to another molecule through an alkylene linker. As used herein, these terms also include rings that contain a double bond or two, as long as the ring is not aromatic.

“Aromatic” or “aryl” substituent or moiety refers to a monocyclic or fused bicyclic moiety having the well-known characteristics of aromaticity; examples of aryls include phenyl and naphthyl. Similarly, “heteroaromatic” and “heteroaryl” refer to such monocyclic or fused bicyclic ring systems which contain as ring members one or more heteroatoms. Suitable heteroatoms include N, O, and S, inclusion of which permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl, and fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-14 ring member atoms. Typically, monocyclic heteroaryls contain 5-6 ring members, and bicyclic heteroaryls contain 8-10 ring members.

Aryl and heteroaryl moieties can be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halogens (F, Cl, Br, I), OR, NR₂, SR, SO₂R, SO₂NR₂, NRSO₂R, NRCONR₂, NRC(O)OR, NRC(O)R, CN, C(O)OR, C(O)NR₂, OC(O)R, C(O)R, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group can be further substituted with the groups described herein as suitable for each type of such substituents or for each component of the substituent. Thus, for example, an arylalkyl substituent can be substituted on the aryl portion with substituents described herein as typical for aryl groups, and it can be further substituted on the alkyl portion with substituents described herein as typical or suitable for alkyl groups.

“Optionally substituted,” as used herein, indicates that the particular group being described can have one or more hydrogen substituents replaced by a non-hydrogen substituent. In some optionally substituted groups or moieties, all hydrogen substituents are replaced by a non-hydrogen substituent (e.g., a polyfluorinated alkyl such as trifluoromethyl). If not otherwise specified, the total number of such substituents that can be present is equal to the number of H atoms present on the unsubstituted form of the group being described. Where an optional substituent is attached via a double bond, such as a carbonyl oxygen or oxo (═O), the group takes up two available valences, so the total number of substituents that may be included is reduced according to the number of available valences.

As used herein, unless specified otherwise, the term “amino group” includes primary, secondary, and tertiary amino groups.

Covalent attachment of amidated pectins to solid supports can be achieved in any suitable manner, such as by reacting the polyamine-amidated pectin with a solid support that comprises amine-reactive groups, for example, an epoxide, aldehyde, ketone, or activated ester. Amidated pectins comprising a primary or a secondary amino group can also be attached to a solid support, e.g., an amino-modified solid surface, by crosslinking. As used herein, crosslinking means the process of chemically joining two or more molecules by a covalent bond. In some instances, a crosslinking agent can be used to attach an amidated pectin to a solid support thereby forming pectin-modified solid supports. As used herein, a crosslinking agent (or crosslinker) is a molecule that contains two or more reactive ends capable of chemically attaching to specific functional groups (such as primary amines, carboxyls, sulfhydryls, etc.) on molecules and/or solid supports. Methods of covalently linking molecules containing amino groups to functionalized surfaces and solid supports are known in the art.

In some embodiments, the amidated pectins of the invention are covalently attached to the solid supports via an amide bond, e.g., an amide bond formed between a carboxy group of the solid support and an amino group of the amidated pectin. Formation of the amide bond can be carried out by any suitable methods. For example, amidated pectin comprising one or more primary amino groups can be reacted with a substrate comprising one or more carboxylic acid groups in the presence of a suitable coupling agent. Non-limiting examples of suitable coupling agents include carbodiimide coupling agents such as DCC and EDCI, phosphonium and imonium type reagents such as BOP, PyBOP, PyBrOP, TBTU, HBTU, HATU, COMU, and TFFH. In some preferred embodiments, the carboxylic acid group of the solid substrate can be converted to an activated ester and then subsequently reacted with an amino group of the amidated pectin.

In some embodiments, the solid supports comprise an amidated pectin having one or more units represented by any one of Formulae (II)-(VIII), wherein the amidated pectin is covalently attached to the solid support.

In another aspect, provided herein is a method for isolation of a nucleic acid from a nucleic-acid containing sample, comprising:

(a) contacting the sample with a solid support disclosed herein thereby binding the nucleic acid to the solid support;

(b) optionally washing the nucleic acid bound to the solid support; and

(c) eluting the nucleic acid from the solid support by contacting the nucleic acid bound to the solid support with an eluting reagent.

### Lysis Solutions

In some embodiments, the nucleic acid-containing sample is contacted with a lysis solution prior to contacting with the solid support, thereby lysing the cells contained in the sample and releasing the nucleic acids into solution. After the sample is lysed, the nucleic acids can be bound to a solid substrate such as silica or glass substrate covalently modified with the amidated pectins described herein. In some embodiments, the solid support is incorporated into an automated cartridge, such as a GenXpert® cartridge. After the binding, the supernatant is then removed, and the nucleic acids are eluted from the substrate with an elution buffer, for example, an alkali solution as described above. The eluate may then be processed in the cartridge to detect target genes of interest. In some embodiments, the eluate is used to reconstitute at least some of the PCR reagents, which are present in the cartridge as lyophilized particles. In some embodiments, the PCR uses Taq polymerase with hot start function, such as AptaTaq (Roche, Switzeland).

In some embodiments, the lysis solution comprises a chaotropic agent, such as guanidinium thiocyanate, guanidinium hydrochloride, alkali perchlorate, alkali iodide, urea, formamide, and combinations thereof. In some embodiments, the lysis solution comprises a salt. Preferably, the salt is sodium chloride or calcium chloride.

In some embodiments, the methods disclosed herein do not require the use of a chaotropic reagent or high salt concentration to bind nucleic acid to the solid support of the invention.

In some embodiments, the sample is lysed by contacting the sample with a lysis buffer prior to addition of the polysaccharide reagent solution and subsequent precipitation of nucleic acids. In some embodiments, the lysing reagent is added to the solution of nucleic acid-precipitating polysaccharide agent. In some embodiments, the polysaccharide reagents described herein are dissolved in the lysis solution. In some instances, the lysis solution comprises one or more proteases. Suitable proteases include, but are not limited to serine proteases, threonine proteases, cysteine proteases, aspartate proteases, metalloproteases, glutamic acid proteases, metalloproteases, and combinations thereof. Illustrative suitable proteases include, but are not limited to proteinase k (a broad-spectrum serine protease), subtilysin trypsin, chymotrypsin, pepsin, papain, and the like. Using the teaching and examples provided herein, other proteases will be available to one of skill in the art.

In some embodiments, the methods described herein are used for isolating a nucleic acid (e.g., a DNA, an RNA) from a fixed paraffin-embedded biological tissue sample according any of the methods described herein, subjecting the precipitated nucleic acid to amplification using a pair of oligonucleotide primers capable of amplifying a region of a target nucleic acid, to obtain an amplified sample; and determining the presence and/or quantity of the target nucleic acid. In some embodiments, the target nucleic acid is a DNA (e.g., a gene). In some embodiments, the target nucleic acid is an RNA (e.g., an mRNA, a non-coding RNA, and the like). In some embodiments, the nucleic acids isolated using the methods described herein are well suited for use in diagnostic methods, prognostic methods, methods of monitoring treatments (e.g., cancer treatment), and the like. Accordingly, in some illustrative, non-limiting embodiments, the nucleic acids extracted from fixed paraffin-embedded samples (e.g., from FFPET samples) can be used to identify the presence and/or the expression level of a gene, and/or the mutational status of a gene. Such methods are particularly well suited to identification of the presence, and/or expression level, and/or mutational status of one or more cancer markers. Accordingly, in some embodiments, the nucleic acids isolated using the methods described herein are utilized to detect the presence, and/or copy number, and/or expression level, and/or mutational status of one or more cancer markers.

### Washing and Elution

The detection and isolation methods disclosed herein can optionally include a washing step, i.e., the precipitated nucleic acid can be optionally washed on solid support for example, to remove components of the lysis buffer. Typically, a concentrated, e.g., precipitated nucleic acid is dissolved prior to detection. In some embodiments, the concentrated nucleic acid is dissolved in a buffer compatible with PCR reactions.

In some embodiments, for example, when a polyamine-modified polysaccharide is used to precipitate the nucleic acid, the precipitated nucleic acid can be eluted from the polyamine by contacting with a suitable eluting agent. In some embodiments, the eluting agent comprises ammonia or an alkali metal hydroxide. In some embodiments, the eluting agent has a basic pH. In some embodiments, the eluting agent has a pH of about 9 to about 12, about 9.5 to about 12, about 10 to about 12, or about 9 to about 11. Preferably, the pH of the eluting agent is above 10. Preferably, the eluting agent comprises ammonium hydroxide, NaOH, or KOH in a concentration sufficient for disrupting the binding of the nucleic acid with the polysaccharide agent. Exemplary eluting agents comprise 1% ammonia, 15 mM KOH, or 15 mM NaOH.

In some embodiments, the eluting agent comprises a polyanion. In some embodiments, the polyanion is a polymer comprising a plurality of anionic groups. In some embodiments, the anionic groups are phosphate, phosphonate, sulfate, or sulfonate groups, or combinations thereof. In some embodiments, the polyanion is a polymer negatively charged at pH above about 7. Both synthetic polyanions and naturally occurring polyanions can be used in the methods disclosed herein. In some embodiments, the polyanion is carrageenan. In other embodiments, the polyanion is a carrier nucleic acid. A carrier nucleic acid, as used herein, is a nucleic acid which does not interfere with the subsequent detection of the concentrated nucleic acid, for example, by PCR. Exemplary carrier nucleic acids include poly rA, poly dA, herring sperm DNA, salmon sperm DNA, and others well known to persons of skilled in the art. In some embodiments, the eluting agent comprises carrageenan and an alkali metal hydroxide, for example, NaOH or KOH.

### Nucleic Acids

In some embodiments, the methods described herein are used to isolate nucleic acids from nucleic acid-containing solutions. The nucleic acid-containing solutions can be obtained by lysis from a nucleic-acid containing material. The nucleic-acid containing material is typically selected from the group comprising blood, tissue biopsy such as paraffin-embedded tissue, smear preparations, bacterial cultures, viral cultures, urine, semen, cell suspensions and adherent cells, PCR reaction mixtures, and in vitro nucleic acid modification reaction mixtures. The nucleic acid-containing material may comprise human, bacterial, fungal, animal, or plant material. In other embodiments, the nucleic acid-containing solution can be obtained from a nucleic acid modification reaction or a nucleic acid synthesis reaction. In other embodiments, the nucleic acid-containing solution can be obtained from a nucleic acid modification reaction or a nucleic acid synthesis reaction.

As used herein, the term “nucleic acid” refers to any synthetic or naturally occurring nucleic acid, such as DNA or RNA, in any possible configuration, i.e., in the form of double-stranded nucleic acid, single-stranded nucleic acid, aptamer, or any combination thereof. The nucleic acid can be DNA, such as genomic DNA. The nucleic acid may also be RNA, such as total RNA. The nucleic acid can be single-stranded or double-stranded nucleic acid, such as short double-stranded DNA fragments. The nucleic acid can be a synthetic nucleic acid. In some embodiments, the nucleic acid is a circulating nucleic acid.

The nucleic acids isolated using the methods and solids supports described herein are of suitable quality to be amplified to detect and/or to quantify one or more target nucleic acid sequences in the sample. The nucleic isolation methods and solid supports described herein are applicable to use in basic research aimed at the discovery of gene expression profiles relevant to the diagnosis and prognosis of disease. The methods are also applicable to the diagnosis and/or prognosis of disease, the determination particular treatment regiments, and/or monitoring of treatment effectiveness.

In some embodiments, the methods described herein are used to precipitate nucleic acids from nucleic acid-containing samples. The nucleic-acid containing material can be selected from the group comprising blood, serum, tissue biopsy such as paraffin-embedded tissue, oral fluids, smear preparations, bacterial cultures, viral cultures, urine, semen, cell suspensions and adherent cells, PCR reaction mixtures, and in vitro nucleic acid modification reaction mixtures. The nucleic acid-containing material may comprise human, animal, or plant material. In some embodiments, the nucleic acid is in solution. The nucleic acid-containing solutions include solution of extracellular nucleic acids and solutions obtained by lysis of a nucleic-acid containing cells. In other embodiments, the nucleic acid-containing solution can be obtained from a nucleic acid modification reaction or a nucleic acid synthesis reaction.

### Amplification Methods

The methods described herein simplify isolation of nucleic acids from biological samples and efficiently produce isolated nucleic acids well-suited for use in RT-PCR systems. In some embodiments, the nucleic acids isolated from a nucleic acid-containing sample using the methods described herein can be detected by any suitable known nucleic acid detection method. While in some embodiments the extracted nucleic acids are used in amplification reactions, other uses are also contemplated. Thus, for example, the isolated nucleic acids or their amplification product(s) can be used in various sequencing or hybridization protocols including, but not limited to nucleic acid-based microarrays and next generation sequencing.

In an aspect, provided herein is a method for detecting a nucleic acid, comprising:

(a) contacting a nucleic acid-containing sample with a solid support disclosed herein thereby binding the nucleic acid to the solid support;

(b) optionally washing the nucleic acid bound to the solid support;

(c) eluting the nucleic acid from the solid support by contacting the nucleic acid bound to the solid support with an eluting reagent; and

(d) detecting the nucleic acid.

In some embodiments, the detection method comprises nucleic acid amplification. Suitable non-limiting exemplary amplification methods include polymerase chain reaction (PCR), reverse-transcriptase PCR, real-time PCR, nested PCR, multiplex PCR, quantitative PCR (Q-PCR), nucleic acid sequence based amplification (NASBA), transcription-mediated amplification (TMA), ligase chain reaction (LCR), rolling circle amplification (RCA), and strand displacement amplification (SDA).

In some embodiments, the amplification method comprises an initial denaturation at about 90° C. to about 100° C. for about 1 to about 10 min, followed by cycling that comprises denaturation at about 90° C. to about 100° C. for about 1 to about 30 seconds, annealing at about 55° C. to about 75° C. for about 1 to about 30 seconds, and extension at about 55° C. to about 75° C. for about 5 to about 60 seconds. In some embodiments, for the first cycle following the initial denaturation, the cycle denaturation step is omitted. The particular time and temperature will depend on the particular nucleic acid sequence being amplified and can readily be determined by a person of ordinary skill in the art.

In some embodiments, the isolation and detection of a nucleic acid is performed in an automated sample handling and/or analysis platform. In some embodiments, commercially available automated analysis platforms are utilized. For example, in some embodiments, the GeneXpert system (Cepheid, Sunnyvale, Calif.) is utilized. However, the present invention is not limited to a particular detection method or analysis platform. One of skill in the art recognizes that any number of platforms and methods may be utilized.

The GeneXpert system utilizes a self-contained, single use cartridge. Sample extraction, amplification, and detection of a nucleic acid can all be carried out within this self-contained “laboratory in a cartridge.” See e.g., U.S. Pat. No. 6,374,684 which is herein incorporated by reference in its entirety. Components of the cartridge include, but are not limited to, processing chambers containing reagents, filters, and capture technologies useful to extract, purify, and amplify target nucleic acids. A valve enables fluid transfer from chamber to chamber and contains nucleic acids lysis and filtration components. An optical window enables real-time optical detection. A reaction tube enables very rapid thermal cycling. In some embodiments, the GenXpert system includes a plurality of modules for scalability. Each module includes a plurality of cartridges, along with sample handling and analysis components.

### Solid Phases for Chromatography

In some embodiments, disclosed herein are separating materials for chromatography comprising a solid support having a polysaccharide bonded thereto. In some embodiments, the polysaccharide is a polyuronic acid or an amidated pectin. In some embodiments, the polysaccharide is an amidated pectin adsorbed on the surface of the solid support. In other embodiments, amidated pectins are immobilized on the surface of the solid support covalently, non-covalently, or via a combination of covalent bonds and non-covalent interactions.

In some embodiments, the separating materials comprise a polysaccharide bonded to a solid support wherein the polysaccharide comprises one or more units represented by Formula II:

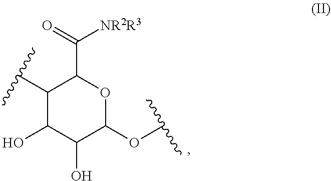

an isomer, a salt, a tautomer, or a combination thereof, wherein

R² and R³ are independently selected from H, optionally substituted C₁-C₆ alkyl, optionally substituted C₃-C₆ cycloalkyl, and optionally substituted C₂-C₂₀ heteroalkyl.

In some embodiments, the amidated pectins are the pectins comprising on or more units having the structure of Formulae II-VIII.

Solid supports suitable for the preparation of the separating materials include silica gel and other inorganic materials, such as Al₂O₃ (alumina), TiO₂ (titania), or ZrO₂ (zirconia). Organic polymeric resins can also be used in the preparation of the separating materials disclosed herein. Certain materials with hybrid particle technology (HPT) are suitable for the preparation of the separating materials disclosed herein, for instance, the hybrid organic/inorganic materials such as Waters BEH Technology™ materials. The HPT materials retain key advantages of silica, such as purity mechanical strength, highly spherical shape, ability to tailor particle size, pore diameter, surface area, and surface chemistry. At the same time, such hybrid materials are stable at basic pH, for instance, stable at pH above 8.

Preferably, the solid supports used for the preparation of the separating materials are porous. In some embodiments, the separating materials are porous particles having amidated pectins bonded thereto via covalent bonds or non-covalent interactions, In other embodiments, the separating material is a porous monolithic support having amidated pectins bonded thereto via covalent bonds or non-covalent interactions.

In some embodiments, the solid support used in the preparation of the separating materials disclosed herein is silica gel or silica. Silica is characterized by pore diameter, particle size, and/or specific surface area. Silica gel-based separating materials preferably have a pore diameter from about 30 to about 1000 Angstroms, a particle size from about 2 to about 300 microns, and a specific surface area from about 35 m²/g to about 1000 m²/g. In some embodiments, silica gels have a pore diameter of about 40 Angstroms to about 500 Angstroms, about 60 Angstroms to about 500 Angstroms, about 100 Angstroms to about 300 Angstroms, and about 150 Angstroms to about 500 Angstroms. In some embodiments, the silica gel has a particle size of about 2 to about 25 microns, about 5 to about 25 microns, about 15 microns, about 63 to about 200 microns, and about 75 to about 200 microns; and a specific surface area of about 100 m²/g to about 350 m²/g, about 100 m²/g to about 500 m²/g, about 65 m²/g to about 550 m²/g, about 100 m²/g to about 675 m²/g, and about 35 to about 750 m²/g.

In some embodiments, the chromatographic material according to the present invention comprises magnetic silica particles. Magnetic silica particles comprise a superparamagnetic core coated with a hydrous siliceous oxide adsorptive surface (i.e. a surface having silanol or Si—OH groups). Suitable commercially available magnetic silica particles include MagneSil™ particles available from Promega Corporation (Madison, Wis.).

In some embodiments, the solid support is aluminum oxide. Exemplary aluminum oxide solid supports include, but are not limited to, Brockmann aluminum oxides that are about 150 mesh and 58 angstroms.

In some embodiments, the amidated pectins are chemically bonded to the solid support via a linker. The linker between the solid support and the amidated pectin can comprise an alkylene or a heteroalkylene chain. Preferably, the linker comprises 2-20 carbon atoms and can contain nitrogen and oxygen atoms in addition to carbon atoms. In some embodiments, the linker is an oligoethylene linker, for example, a PEG oligomer.

Preparation of the separating materials can be achieved in any suitable manner. For example, the solid support can be reacted with a surface modifier. As used herein, a surface modifier is a moiety that that imparts some chromatographic functionality to the base solid support. Surface modifiers, such as amidated pectins disclosed herein, can be attached to the base solid support via derivatization reactions, non-covalent coating, or a combination thereof. In some embodiments, the organic group of the base solid support forms a covalent bond with the surface modifier, such as an amidated pectin comprising a reactive group. Such covalent attachment of the amidated pectin can be achieved via a number of mechanisms well known in the art, such as cycloaddition and nucleophilic and electrophilic substitution.

In some embodiments, the base solid support is silica gel comprising silanol groups. Such silica gel solid supports can be reacted with a modifier comprising a silanizing group to obtain the separating materials disclosed herein. For example, silanol groups are surface-modified with a silanizing reagent having the formula XₐR_{b}Si-L-Z, wherein X is Cl, Br, I, C1-C5 alkoxy, dialkylamino, or trifluoromethanesulfonate; a and b are each integers from 0 to 3, wherein the sum of a and b equals 3; R is a C1-C6 straight, branched, or cyclic alkyl; L is an optional C1-C20 alkylene or heteroalkylene linker group which may be optionally substituted; and Z is a functionalizing group.

In some embodiments, Z comprises an amidated pectin. In other embodiments, Z comprises a functional group that can be further functionalized with an amidated pectin, such as an amino, carbonyl, or a carboxyl group. Examples of silanizing agents include amino silanizing agents, such as 3-aminopropyltrimethoxysilane, 3-aminopropyltriethoxysilane, aminoalkylsilatranes, 3-(2-aminoethyl)aminopropyl-triethoxysilane, and 3-(2-aminoethyl)aminopropyltriethoxysilane. The reaction of silica gel with an amino silanizing agent provides in silica gel comprising surface amino groups that can be further modified and/or reacted with amidated pectin comprising one or more reactive groups. In other embodiments, the silica gel is reacted with an amidated pectin derivative that comprises a silica-reactive group, such as a silatrane or trialkoxysilane derivative.

In some embodiments, disclosed herein are columns, capillaries, or cartridges containing, as sorbent or support, solid supports comprising a surface and one or more molecules of amidated pectin bound to the surface.

In some embodiments, the separating materials and chromatography columns disclosed herein are useful for isolation, separation, and purification of nucleic acids, for example, from a biological sample or a chemical reaction mixture. In some embodiments, the separation is achieved by high performance liquid chromatography (HPLC), size exclusion chromatography, or electrophoresis.

In some embodiments, the separating materials disclosed herein are suitable for separation of nucleic acids, including but not limited to dsDNA, ssDNA, RNA, and their hybrids. Elution of the nucleic acids off the separating material and their separation can be achieved by increasing the ionic strength of the eluent mobile phase or by increasing the concentration of eluting agent, stepwise or in a gradient manner. The mobile phase can optionally contain an organic solvent suitable for HPLC separations, such as acetonitrile or methanol. The increase of ionic strength can be achieved by increasing concentration of a suitable salt, such as sodium chloride or guanidinium salts.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The present invention is further illustrated by the following Examples, which are intended merely to further illustrate and should not be construed as limiting.

### EXAMPLES

### Example 1: Preparation of Amidated Pectin-Modified Solid Supports (EDC Route)

### A. Preparation of Amidated Pectin-Modified Beads

All reagents were from commercial sources unless indicated otherwise.

Pectins amidated with spermine were prepared according to the procedure described below. Other amidated pectins were prepared in a similar manner.

(A) Apple pectin (2.5 g) was added in portions to 250 mL deionized water with magnetic stirring until it all dissolved. To this solution, 2.5 mL of 5M NaOH was added and stirred for 20 min, followed by 1M HCl until pH stabilized at ˜4.5 (˜12 mL of WI HCl was then added). 1-Ethyl-3-(3-dimethylaminopropyl)-carbodiimide hydrochloride (EDC.HCl, 2.5 g) was then added and 0.75 g of N-hydroxysuccinimide (NHS) and stirred for 1 hour for activation. Spermine (Sigma, 18.63 g, 7 eq) was then added at once. The solution became gel like, and shaken until everything is dissolved, and incubated further for 20 hours at room temperature.

(B) The reaction mixture from (A) was poured into 500 mL MeOH with stirring, forming a gel like precipitate. The mixture was then stirred for 30 min and filtered through a 500 mL plastic disposable filter with polyethylene frit (Opti-Chem, OP-6602-18). The gel-like cake that collected was then rinsed with methanol (100 mL), and allowed to further filter overnight forming dried brown gel chunks. The material was then washed with another 150 mL MeOH and dried in vacuum oven for 18 hours at 50 C. The hard pellet that formed was crushed to powder in a mortar with a pestle.

(C) Wash

Materials:
A. Acidic wash. In a 1000 mL bottle the following mixture was prepared: IPA (550 mL, graduated cylinder), DI water (345 mL) and conc. HCl (105 mL)
B. Neutral wash. In a 1000 mL flask the following mixture was prepared: 590 mL IPA with 410 mL DI water

The product from step (B) was loaded into 125 mL flasks and 110 mL of a wash solution was added to the powdery material. The suspension was stirred at RT for 30 min and filtered on a glass funnel and washed 5×15-20 mL of acid wash followed by 5×15-20 mL of neutral wash followed by 2×35 mL MeOH. The material was further air dried for 60 min and then at 0.15 mbar for 17 h.

### B. Preparation of Amidated Pectin-Modified Beads

The following solid support (bead) materials were modified with amidated pectins according to the procedure described below:

Silica Microspheres, Carboxyl, 1.0 μm (Polysciences, Warrington, Pa., 24754-1)

Carboxyl-polystyrene Particles, 5.11 μm (Spherotec, Germany, CP-50-10)

NETS-Activated Sepharose 4 Fast Flow (Sepharose beads, GE healthcare, Chicago, Ill., 17-0906-01); and

Carboxyl-modified magnetic beads, 5.7 μm (Spherotec, Germany).

For Sepharose beads, the NHS-activated bead form was used, and the EDC/NHS activation step was omitted. Hydrolyzed NHS-Sepharose beads were used for non-modified bead measurement.

In this Example, a procedure is provided for functionalization of carboxyl modified beads with an amine-containing amidated pectin, such as the product from Example 1.

Polystyrene beads (˜5 micron, 2 mL of 5 wt % suspension) modified with carboxyl groups (Spherotec, CP-50-10) were diluted with deionized (DI) water (4 mL) and sonicated for 15 min. To the bead suspension, 40 mg of EDC.HCl and 40 mg of NHS were added. The suspension was stirred for 24 hours for activation, briefly centrifuged at 4000 rpm for 5 min and the supernatant decanted. Beads were resuspended in 5 mL DI water, and to this was added a 1% solution of amidated pectin (5 mL). Amidated pectin solution was prepared by amidated pectin in DI water for 18 hours, followed by centrifugation at 9000 rpm for 30 min to remove any dissolved material. The resulting suspension was stirred for 18 hours, then centrifuged at 9000 rpm for 30 min, diluted with 45 mL of water and rinsed in the same manner. The process was repeated with 0.1 M NaOH (1×), 0.1 M HCl (1×), and DI water (2×). Beads were resuspended in 5 mL DI H₂O, sonicated for 30 min, and concentration measured by weighing the amount of beads left after drying a 150 μL aliquot under vacuum in a SpeedVac.

### Example 2: Preparation of Amidated Pectin-Modified Solid Supports (Reductive Amination Route)

In this example, a general procedure is provided for the modification of polysaccharides, e.g., pectins, with various polyamines through oxidation followed by reductive amination.

(A). Oxidation. Apple pectin (2.5 g) was added in portions to 250 mL deionized water with magnetic stirring until it has all dissolved. To this, was added potassium periodate (2.43 g) was added in portions with stirring and was left stirring for 18 h. Reaction mixture was then dialyzed against water through 8 kDa MWCO dialysis tubing over 3 days. The resulting desalted polymer was subsequently lyophilized to give oxidized pectin as an off-white solid. The concentration of aldehydes can be readily measured via hydroxylamine titration (e.g., as described in Zhao, H.; Heindel, N. D. J. Pharm. Res. 8(3), 400-402.) Aldehyde content was determined to be 4.9 mmol/g (˜1 eq aldehyde per polymer unit).

(B). Reductive amination. The oxidized pectin from step A (1.0 g) was suspended in 100 mL of deionized water, spermine (1.32 g, 1.25 eq) was added, and the mixture was stirred for 18 h at room temperature. Sodium borohydride pellet (1.0 g) was added to the reaction, and the mixture was stirred for 18 hr. The reaction mixture was then dialyzed against water through 8 kDa MWCO dialysis tubing over three days and subsequently lyophilized to yield 200 mg of amidated pectin as off-white, fluffy solid.

The product from the reactions above was used in modification of solid supports as described above in Example 1.

### Example 3: Assessment of Nucleic Acid Capture by Modified Beads on Filter

This experiment demonstrated that exemplary solid supports, e.g., amidated pectin-modified beads prepared as described in Example 1 can capture DNA or RNA on a filter.

Materials

The following materials were used in the example: Genomic DNA (Promega Cat#G3041 ˜202 ng/μL); RNA Control (Life Tech Cat#4307281, 50 ng/μL); Quantitative Fluorescent Picogreen DNA dye (Thermo); Quantitative Fluorescent Ribogreen RNA dye (Thermo); Biotek fluorimeter and black assay plates suitable for fluorometric quantification of nucleic acids; Calibrated pipette and pipette tips; 1×TE buffer (as per manufacturer's instructions Thermo: EnzChek® Reverse Transcriptase Assay Kit, P/N E22064), or 20 mM Tris prepared with pH˜8.5; Whatman GF/F filters and Pall Supor 0.2 micron filters; Filter holder

Method

Test solutions of DNA or RNA in 1×TE buffer were prepared at desired final concentration (e.g 100 ng/mL). To the test solutions, DNA or RNA in TE with modified beads was added. As a control, a DNA or RNA solution was prepared that has no added beads. Exemplary test solutions:
TE buffer with nucleic acid with 0.1-1.5 mg of modified beads;
TE buffer with nucleic acid (negative control) no beads;
TE buffer with nucleic acid, no beads, unfiltered.

A 1 mL sample of a nucleic acid solution was mixed with modified beads for 15 seconds to facilitate mixing and binding of nucleic acids to the bead surface. The samples were aspirated into 1 mL syringe; passed through a GF/F or other filter of interest using a syringe filter device or premade filters. The eluent was collected into 2 mL Eppendorf tube. As the captured nucleic acid was retained on beads on filter, the amount of the captured nucleic acid can be assessed indirectly by lack of nucleic acid in eluent as follows.

A standard curve for DNA or RNA was prepared as directed by the manufacturer's instructions; 500 μL of each standard and a blank in a total of 8 tubes were prepared. Working dye solutions were prepared by diluting dye 1:200 in TE buffer and protected from light. The fluorescence of the standard curve samples and each eluent sample were measured as per the manufacturer's instructions in the Biotek plate reader. The standard curve was used to calculate the concentration of nucleic acid in the eluent samples and to calculate the percentage capture relative to theoretical concentration. Test samples were compared to the 100% unfiltered control to determine the percentage recovery of nucleic acid. A no bead control sample was filtered to assess background filter capture, which was minimal. The 100% control was not filtered.

Tables 1-5 shows results from filtration experiments demonstrating that the solid supports modified with amidated pectin can efficiently capture nucleic acids.

TABLE 1
hgRNA and hgDNA capture by modified glass beads on Pall Supor 0.2 micron filter.

| Bead | | 0.1 mg/mL | 0.25 mg/mL | 0.5 mg/mL | 1 mg/mL | 1.5 mg/mL |
|---|---|---|---|---|---|---|
| Type | Modifier | bead | bead | bead | bead | bead |
| | | hgRNA captured (% of 100 ng) | | | | |
| glass μm | none | 0% | 12% | 6% | 8% | 1% |
| glass μm | spermine | 11% | 7% | 12% | 18% | 9% |
| glass μm | spermidine | 9% | 12% | 15% | 10% | 19% |
| glass μm | pectin-spermine | 11% | 8% | 12% | 10% | 19% |
| glass μm | pectin-spermidine | 12% | 9% | 15% | 22% | 25% |
| glass μm | Pectin | 12% | 16% | 14% | 25% | 27% |
| | ethylenediamine | | | | | |
| None | N/A | 0% | 0% | 0% | 0% | 0% |
| | | hgDNA capture (% of 100 ng) | | | | |
| glass μm | None | 13% | 58% | 64% | 80% | 85% |
| glass μm | spermine | 49% | 64% | 72% | 84% | 86% |
| glass μm | spermidine | 48% | 68% | 72% | 81% | 85% |
| glass μm | pectin-spermine | 37% | 55% | 68% | 80% | 86% |
| glass μm | pectin-spermidine | 53% | 51% | 62% | 78% | 75% |
| glass μm | pectin- | 45% | 66% | 65% | 77% | 75% |
| | ethylenediamine | | | | | |
| None | N/A | 16% | 16% | 16% | 16% | 16% |

TABLE 2
hgRNA and hgDNA capture by modified sepharose beads on a Whatman GF/F filter.

| Bead | | 0.1 mg/mL | 0.25 mg/mL | 0.5 mg/mL | 1 mg/mL | 1.5 mg/mL |
|---|---|---|---|---|---|---|
| Type | Modifier | bead | bead | bead | bead | bead |
| | | hgRNA captured (% of 100 ng) | | | | |
| Sepharose | None | 16% | 13% | 18% | 33% | 34% |
| Sepharose | Spermine | 34% | 51% | 52% | 60% | 71% |
| Sepharose | spermidine | 7% | 30% | 41% | 62% | 66% |
| Sepharose | pectin-spermine | 0% | 14% | 38% | 55% | 52% |
| Sepharose | pectin-spermidine | 38% | 54% | 65% | 65% | 64% |
| Sepharose | pectin- | 23% | 14% | 19% | 35% | 42% |
| | ethylenediamine | | | | | |
| None | N/A | 38% | 38% | 38% | 38% | 38% |
| | | hgDNA capture (% of 100 ng) | | | | |
| Sepharose | None | 20% | 7% | 8% | 19% | 12% |
| Sepharose | Spermine | 47% | 84% | 91% | 97% | 93% |
| Sepharose | spermidine | 52% | 40% | 36% | 38% | 47% |
| Sepharose | pectin-spermine | Not | 15% | 37% | 68% | 75% |
| | | detected | | | | |
| Sepharose | pectin-spermidine | 5% | 32% | 44% | 64% | 71% |
| Sepharose | pectin- | 18% | 15% | 26% | 13% | 31% |
| | ethylenediamine | | | | | |
| None | N/A | 38% | 38% | 38% | 38% | 38% |

TABLE 3
hgRNA and hgDNA capture by modified polystyrene beads on a Whatman GF/F filter.

| Bead | | 0.1 mg/mL | 0.25 mg/mL | 0.5 mg/mL | 1 mg/mL | 1.5 mg/mL |
|---|---|---|---|---|---|---|
| Type | Modifier | bead | bead | bead | bead | bead |
| | | hgRNA captured (% of 100 ng) | | | | |
| Polystyrene | none | 35% | 35% | 47% | 41% | 21% |
| Polystyrene | spermine | 42% | 59% | 58% | 74% | 85% |
| Polystyrene | spermidine | 42% | 37% | 44% | 70% | 76% |
| Polystyrene | pectin-spermine | 29% | 41% | 63% | 83% | 86% |
| Polystyrene | pectin-spermidine | 38% | 56% | 72% | 86% | 81% |
| Polystyrene | pectin- | 8% | 12% | 28% | 39% | 46% |
| | ethylenediamine | | | | | |
| None | N/A | 38% | 38% | 38% | 38% | 38% |
| | | hgDNA capture (% of 100 ng) | | | | |
| Polystyrene | none | 21% | 24% | 32% | 36% | 17% |
| Polystyrene | spermine | 98% | 99% | 94% | 100% | 100% |
| Polystyrene | spermidine | 16% | 17% | 26% | 35% | 35% |
| Polystyrene | pectin-spermine | 35% | 53% | 68% | 84% | 90% |
| Polystyrene | pectin-spermidine | 47% | 74% | 82% | 94% | 89% |
| Polystyrene | pectin- | 29% | 35% | 23% | 30% | 27% |
| | ethylenediamine | | | | | |
| None | N/A | 38% | 38% | 38% | 38% | 38% |

TABLE 4
hgRNA and hgDNA capture by modified polystyrene beads on Pall Supor 0.2 micron filter.

| Bead | | 0.1 mg/mL | 0.25 mg/mL | 0.5 mg/mL | 1 mg/mL | 1.5 mg/mL |
|---|---|---|---|---|---|---|
| Type | Modifier | bead | bead | bead | bead | bead |
| | | hgRNA captured (% of 100 ng) | | | | |
| Sepharose | None | 16% | 17% | 18% | 17% | 18% |
| Sepharose | spermine | 29% | 41% | 51% | 65% | 83% |
| Sepharose | spermidine | 26% | 37% | 49% | 69% | 69% |
| Sepharose | pectin-spermine | 30% | 38% | 42% | 70% | 90% |
| Sepharose | pectin-spermidine | 15% | 11% | 13% | 28% | 28% |
| Sepharose | pectin- | 19% | 23% | 25% | 35% | 50% |
| | ethylenediamine | | | | | |
| None | N/A | 10% | 10% | 10% | 10% | 10% |
| | | hgDNA capture (% of 100 ng) | | | | |
| Sepharose | None | 15% | 18% | 17% | 16% | 16% |
| Sepharose | spermine | 10% | 34% | 27% | 42% | 49% |
| Sepharose | spermidine | 15% | 18% | 29% | 52% | 56% |
| Sepharose | pectin-spermine | 19% | 23% | 48% | 68% | 83% |
| Sepharose | pectin-spermidine | 17% | 21% | 37% | 35% | 51% |
| Sepharose | pectin- | 18% | 15% | 26% | 13% | 31% |
| | ethylenediamine | | | | | |
| None | N/A | 26% | 26% | 26% | 26% | 26% |

TABLE 5
hgRNA and hgDNA capture by modified polystyrene beads on Pall Supor 0.2 micron filter.

| Bead | | 0.1 mg/mL | 0.25 mg/mL | 0.5 mg/mL | 1 mg/mL | 1.5 mg/mL |
|---|---|---|---|---|---|---|
| Type | Modifier | bead | bead | bead | bead | bead |
| | | | hgRNA captured (% of 100 ng) | | | |
| Polystyrene | none | 0% | 0% | 0% | 8% | 8% |
| Polystyrene | spermine | 33% | 61% | 88% | 104% | 105% |
| Polystyrene | spermidine | 15% | 21% | 19% | 24% | 17% |
| Polystyrene | pectin- | 35% | 40% | 67% | 87% | 93% |
| | spermine | | | | | |
| Polystyrene | pectin- | 12% | 36% | 48% | 66% | 77% |
| | spermidine | | | | | |
| Polystyrene | pectin- | 18% | 11% | 11% | 7% | 9% |
| | ethylenediamine | | | | | |
| None | N/A | 10% | 10% | 10% | 10% | 10% |
| | | | hgDNA capture (% of 100 ng) | | | |
| Polystyrene | none | 10% | 10% | 24% | 17% | 22% |
| Polystyrene | spermine | 21% | 44% | 54% | 79% | 95% |
| Polystyrene | spermidine | 18% | 36% | 18% | 32% | 35% |
| Polystyrene | pectin- | 33% | 43% | 57% | 82% | 90% |
| | spermine | | | | | |
| Polystyrene | pectin- | 37% | 58% | 70% | 83% | 91% |
| | spermidine | | | | | |
| Polystyrene | pectin- | 27% | 27% | 17% | 20% | 30% |
| | ethylenediamine | | | | | |
| None | N/A | 26% | 26% | 26% | 26% | 26% |

### Example 4. Extraction of Nucleic Acid from Urine and Stool

This experiment demonstrates that the solid supports disclosed herein can be used to extract nucleic acids from stool and urine samples, and the isolated DNA can be detected by PCR amplification.

Preparation of Urine or Stool Samples

Fragmented MTB DNA (fMTB DNA 200-400 bp) was spiked into urine or stool samples of various volumes as indicated below. Controls for this experiment were prepared by spiking the same amount of fMTB DNA directly into a separate RT-PCR reaction in order to have a comparison indicative of 100% extraction and recovery efficiency.

Extraction of Fragmented MTB DNA from Urine or Stools Using Exemplary Microparticles Modified with Amidated Pectins.

A 1-10 mL urine/stool sample was added into an appropriately sized centrifuge tube or Eppendorf tube. The exemplary microparticles modified amidated pectins were added to the sample. An optimal amount to add depended on bead lot, sample type, and sample volume chosen for each experiment. The sample was mixed thoroughly, optionally allowed to incubate up to 60 min to increase nucleic acid binding, and thereafter spun down in a table top centrifuge at high speed for two minutes in order to sediment the microparticles. The supernatant was decanted carefully so as to not disturb the microparticle pellet. One mL of water was used to wash the bead pellet, mixed gently to wash the pellet, and spun down in a table top centrifuge at high speed for two minutes in order to sediment the microparticles. The supernatant was decanted carefully so as to not disturb the microparticle pellet. 100 μL of low salt elution buffer was added to the bead pellet comprised of 10 mM KOH with 0.01% i-carrageenan (Sigma). The pellet was mixed gently and optionally incubated up to 60 min to increase elution from the microparticles. The supernatant which contains the eluted nucleic acids was removed carefully so as to not disturb the bead pellet. The eluent was then used directly in a RT-PCR reaction. PCR was performed as described for the Xpert MTB/RIF Ultra Assay by Chakravorty et al. mBio, July/August 2017 Volume 8 Issue 4 e00812-17

The results are shown in Tables 6-8 below.

TABLE 6
PCR analysis of DNA extracted from 10 mL of urine sample.
Performance of microparticles modified with pectins amidated with spermine (EDC
coupling or reductive amination) in extracting MTB DNA from 10 mL urine is shown.
ΔCt are calculated as the resulting extract Ct difference from a 100% spike in control.

| | | | | | | μL | ΔCt |
|---|---|---|---|---|---|---|---|
| Pectin | Base bead | EDC/ | | | | particles | from |
| Modifier/ | (2.5 mL of | NHS | Compound | Time, | | (2.5%) | 100% |
| Procedure | 2.5% soln) | (mg) | (mg) | days | Wash | per mL | control |
| Spermine/ | carboxylated | 50/200 | 10 | 1 | 10 mM | 10-20 | 1.5-2.5 |
| EDC | iron oxide | | | | KOH, | | |
| coupling | nanoparticles | | | | 0.01% | | |
| | | | | | Tween | | |
| Spermine/ | Thermo | 31/156 | 18.75 | 1 | 0.1 M | 10-15 | 5.5-5.9 |
| EDC | magnetic | | | | HCl, | | |
| coupling | beads | | | | 0.01% | | |
| | (1-4 μm) | | | | Tween | | |
| Spermine/ | Spherotech | 25/100 | 20 | 3 | 10 mM | 10-20 | 1.2-2.8 |
| reductive | magnetic | | | | KOH, | | |
| amination | beads | | | | 0.01% | | |
| | (5.7 μm) | | | | tween | | |
| Spermine/ | Spherotech | 25/100 | 20 | 3 | 10 mM | 20-40 | 2.5-2.8 |
| reductive | magnetic | | | | KOH, | | |
| amination | beads | | | | 0.01% | | |
| | (5.7 μm) | | | | tween | | |
| Spermine/ | Spherotech | 25/50 | 4 | 1 | 10 mM | 15-20 | 2.8-5.3 |
| reductive | magnetic | | | | KOH, | | |
| amination | beads | | | | 0.01% | | |
| | (5.7 μm) | | | | tween | | |

TABLE 7
PCR analysis of DNA extracted from 1 mL urine sample Performance of
microparticles modified with pectins amidated with spermine (EDC
coupling or reductive amination) in extracting MTB DNA from 1 mL
urine is shown. ΔCt are calculated as the resulting extract Ct difference
from a 100% spike in control.

| | | | | | | uL | |
|---|---|---|---|---|---|---|---|
| Pectin | Base bead | EDC/ | Amidated | | | particles | ΔCt from |
| Modifier/ | (2.5 mL of | NHS | Pectin | Time, | | (2.5%) | 100% |
| Procedure | 2.5% soln) | (mg) | (mg) | days | Wash | per mL | control |
| Spermine | Spherotech | 25/100 | 20 | 3 | 20 mM | 10-100 | 1.5-4 |
| reductive | magnetic | | | | KOH, | | |
| amination | beads | | | | 0.01% | | |
| (NaBH₄) | (5.7 μm) | | | | Tween | | |
| Spermine/ | Spherotech | 25/100 | 20 | 3 | 10 mM | 10-100 | 0.7-4.7 |
| reductive | magnetic | | | | KOH, | | |
| amination | beads | | | | 0.01% | | |
| (NaBH₄) | (5.7 μm) | | | | Tween | | |
| Spermine/ | Spherotech | 25/100 | 20 | 3 | 10 mM | 10-100 | 1-3.5 |
| reductive | magnetic | | | | KOH, | | |
| amination | beads | | | | 0.01% | | |
| (NaBH₄) | (5.7 μm) | | | | Tween | | |
| Spermine/ | Spherotech | 25/100 | 20 | 3 | 20 mM | 10-100 | No |
| reductive | magnetic | | | | KOH, | | Ct-3 |
| amination | beads | | | | 0.01% | | |
| (STABH) | (5.7 μm) | | | | Tween | | |

TABLE 8
PCR analysis of DNA extracted from 1 mL stool sample. Different microparticle
modification formulations and their performance in extracting MTB DNA
from 1 mL stool sample. Presence of a reducing agent indicates polymer
modification via reductive amination route. No reducing agent (N/A) indicates
polymer modification via EDC/NHS. ΔCt are calculated as the resulting
extract Ct difference from a 100% spike in control.

| | | | | | | uL | ΔCt |
|---|---|---|---|---|---|---|---|
| Pectin | Base bead | EDC/ | | | | particles | from |
| Modifier/ | (2.5 mL of | NHS | CP | Time; | | (2.5%) | 100% |
| Procedure | 2.5% soln) | (mg) | (mg) | days | Wash | per mL | control |
| Spermine/ | Spherotech | 50/200 | 20 | 1 | 10 mM | 10.00 | 3.2 |
| EDC | magnetic beads | | | | KOH, | | |
| coupling | (2.8 μm) | | | | 0.01% | | |
| | | | | | tween | | |
| Spermine/ | carboxylated | 50/200 | 10 | 1 | 10 mM | 100-200 | 2-2.4 |
| EDC | iron oxide | | | | KOH, | | |
| coupling | nanoparticles | | | | 0.01% | | |
| | | | | | tween | | |
| Spermine/ | Thermo | 31/156 | 6.25 | 1 | 0.1 M | 10 | 1.7 |
| EDC | magnetic beads | | | | HCl, | | |
| coupling | (1-4 μm) | | | | 0.01% | | |
| | | | | | tween | | |
| Spermine/ | Thermo | 31/156 | 12.5 | 1 | 0.1 M | 10 | 0.8 |
| EDC | magnetic beads | | | | HCl, | | |
| coupling | (1-4 μm) | | | | 0.01% | | |
| | | | | | tween | | |
| Spermine/ | Thermo | 31/156 | 18.75 | 1 | 0.1 M | 10 | 0.2 |
| EDC | magnetic beads | | | | HCl, | | |
| coupling | (1-4 μm) | | | | 0.01% | | |
| | | | | | tween | | |
| Spermine/ | Thermo | 31/156 | 25 | 1 | 0.1 M | 10 | 3.7 |
| EDC | magnetic beads | | | | HCl, | | |
| coupling | (1-4 μm) | | | | 0.01% | | |
| | | | | | tween | | |
| Spermine/ | Spherotech | 25/100 | 20 | 3 | 10 mM | 10-50 | 1.7-3.5 |
| reductive | magnetic beads | | | | KOH, | | |
| amination | (5.7 μm) | | | | 0.01% | | |
| (NaBH₄) | | | | | tween | | |
| Spermine/ | Spherotech | 25/100 | 20 | 3 | 10 mM | 10 | 3.8 |
| reductive | magnetic beads | | | | KOH, | | |
| amination | (5.7 μm) | | | | 0.01% | | |
| (NaBH₄) | | | | | tween | | |
| Spermine/ | Spherotech | 25/100 | 5 | 3 | 10 mM | 10 | 3.6 |
| reductive | magnetic beads | | | | KOH, | | |
| amination | (5.1 μm) | | | | 0.01% | | |
| (NaBH₄) | | | | | tween | | |

While illustrative embodiments have been illustrated and described, it will be appreciated that various changes can be made therein without departing from the spirit and scope of the invention.

What is claimed is:

1. A solid support comprising a plurality of modified pectin molecules covalently bound to the solid support.

2. The solid support of claim 1, wherein the modified pectin comprises a plurality of amino groups.

3. The solid support claim 1, wherein the modified pectin is an amidated pectin.

4. The solid support of claim 3, wherein the amidated pectin comprises one or more units represented by Formula: 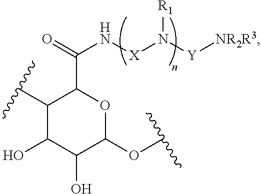
an isomer, a salt, a tautomer, or a combination thereof,
wherein
n is 0-3;
R¹ is H or C₁-C₃ alkyl;
X, at each occurrence, is independently C₂-C₄ alkylene or C₄-C₆ heteroalkylene;
Y is a C₂-C₃ alkylene or C₄-C₆ heteroalkylene; and
R² and R³ are independently H or C₁-C₃ alkyl.

5. The solid support of claim 3, wherein the amidated pectin is a pectin amidated with a C₄-C₂₀ polyamine.

6. The solid support of claim 5, wherein the polyamine is ethylenediamine, putrescine, cadaverine, spermine, or spermidine.

7. The solid support of claim 3, wherein the amidated pectin comprises one or more units having the structure: 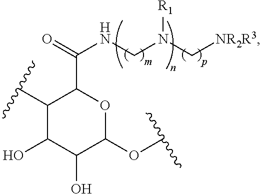
an isomer, a salt, a tautomer, or a combination thereof, wherein
n is 0, 1, 2, or 3;
m is 2, 3, or 4;
p is 2, 3, or 4; and
R¹, R², and R³ are independently H or C₁-C₃ alkyl.

8. The solid support of claim 3, wherein the amidated pectin comprises one or more units having the structure: 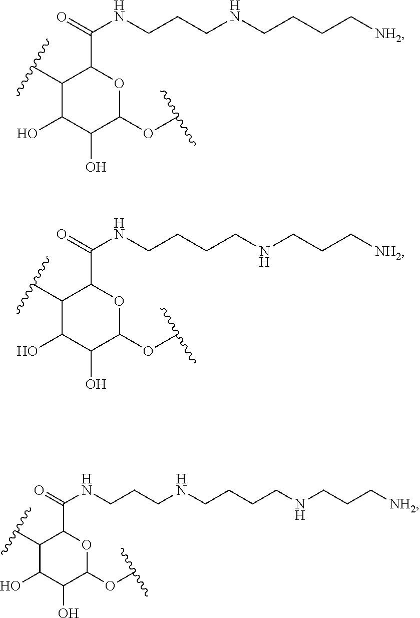 [SMILES: *OC1OC(C(=O)NCCCNCCCCN)C(*)C(O)C1O ; *OC1OC(C(=O)NCCCCNCCCN)C(*)C(O)C1O ; *OC1OC(C(=O)NCCCNCCCCNCCCN)C(*)C(O)C1O]
or
their isomers, salts, or tautomers.

9. The solid support of claim 3, wherein the amidated pectin is amidated citrus pectin or amidated apple pectin.

10. The solid support of claim 3, wherein the amidated pectin has a molecular weight between about 4,000 Da and about 500,000 Da, between about 5,000 Da and about 300,000 Da, between about 100,000 Da and about 300,000 Da, or between about 50,000 Da and about 200,000 Da.

11. The solid support of claim 1, wherein the solid support comprises a material selected from polystyrene, glass, ceramic, polypropylene, polyethylene, silica, zirconia, titania, alumina, polycarbonate, latex, PMMA, zeolite, polyethersulfone, carboxymethylcellulose, and cellulose.

12. The solid support of claim 1, wherein the solid support is a magnetic bead, a glass bead, polystyrene bead, a polystyrene filter, a polycarbonate filter, a polyethersulfone, or a glass filter.

13. A method for isolation of a nucleic acid from a nucleic-acid containing sample, comprising:
(a) contacting the sample with a solid support of any one of claims 1-12 thereby binding the nucleic acid to the solid support;
(b) optionally washing the nucleic acid bound to the solid support; and
(c) eluting the nucleic acid from the solid support with an eluting agent.

14. The method of claim 13, wherein the eluting agent comprises ammonia or an alkali metal hydroxide.

15. The method of claim 13, wherein the eluting agent has a pH of above about 9, above about 10, or above about 11.

16. The method of claim 13, wherein the eluting agent has a pH between about 9 and about 12, between about 9.5 and about 12, between about 10 and about 12, or between about 9 and about 11.

17. The method of claim 13, wherein eluting agent comprises a polyanion.

18. The method of claim 17, wherein the polyanion is carrageenan.

19. The method of claim 17, wherein the polyanion is a carrier nucleic acid.

20. The method of claim 13, wherein the eluting agent comprises carrageenan and KOH.

21. The method of any one of claims 13-20, wherein the method comprises contacting the sample with a lysis solution prior to contacting the sample with the solid support, thereby releasing nucleic acids into solution.

22. The method of claim 21, wherein the lysis solution comprises a chaotropic agent.

23. The method of claim 22, wherein the chaotropic agent is selected from guanidinium thiocyanate, guanidinium hydrochloride, alkali perchlorate, alkali iodide, urea, formamide, or combinations thereof.

24. The method of claim 22, wherein the chaotropic agent is guanidinium thiocyanate or guanidinium hydrochloride.

25. The method of claim 21, wherein the lysis solution comprises a salt.

26. The method of claim 25, wherein the salt is sodium chloride or calcium chloride.

27. The method of claim 21, wherein the lysis solution does not contain a chaotropic agent.

28. The method of claim 21, wherein the lysis solution comprises a buffering agent.

29. The method of claim 28, wherein the buffering agent is Tris.

30. The method of claim 21, wherein the lysis solution comprises a surfactant.

31. The method of claim 21, wherein the lysis solution comprises a defoaming agent.

32. The method of claim 13, wherein contacting the sample with a solid support is done without the presence of a chaotropic reagent.

33. The method of claim 13, wherein the sample is selected from blood, plasma, serum, semen, tissue biopsy, urine, stool, saliva, smear preparation, bacterial culture, cell culture, viral culture, PCR reaction mixture, or in vitro nucleic acid modification reaction mixture.

34. The method of claim 33, wherein the tissue biopsy is a paraffin-embedded tissue.

35. The method of claim 13, wherein the nucleic acid comprises genomic DNA.

36. The method of claim 13, wherein the nucleic acid comprises total RNA.

37. The method claim 13, wherein the nucleic acid comprises microbial nucleic acid or viral nucleic acid.

38. The method of claim 37, wherein the viral nucleic acid is HBV DNA.

39. The method of claim 13, wherein the nucleic acid is a circulating nucleic acid.

40. The method of any one of claims 13-39, wherein the method is performed in an automated cartridge.

41. A method for detecting a nucleic acid in a sample, comprising:
(a) contacting a nucleic acid-containing sample with a solid support of any one of claims 1-12 thereby binding the nucleic acid to the solid support;
(b) optionally washing the nucleic acid bound to the solid support;
(c) eluting the nucleic acid; and
(d) detecting the nucleic acid.

42. The method of claim 41, wherein detecting the nucleic acid comprises amplification of the nucleic acid by polymerase chain reaction (PCR).

43. The method of claim 42, wherein the polymerase chain reaction is a nested PCR, an isothermal PCR, or RT-PCR.

44. A separating material for chromatography comprising a solid support comprising an amidated pectin chemically bonded thereto.

45. The separating material of claim 44, wherein the amidated pectin has one or more units represented by formula: 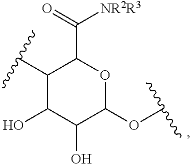
an isomer, a salt, a tautomer, or a combination thereof,
wherein R² and R³ are independently selected from H, optionally substituted C₁-C₆ alkyl, optionally substituted C₃-C₆ cycloalkyl, and optionally substituted C₂-C₂₀ heteroalkyl.

46. The separating material of claim 44 or claim 45, wherein the solid support is silica, alumina, titania, zirconia, or a hybrid silica material.
